(19) United States
(12) Patent Application Publication
(10) Pub. No.: US 2011/0071369 A1
(43) Pub. Date: Mar. 24, 2011

(54) SPOT OR RASH TESTER

(76) Inventor: Janet Lawrence, Birmingham (GB)

(21) Appl. No.: 12/919,369

(22) PCT Filed: Feb. 25, 2009
(86) PCT No.: PCT/GB2009/000509

§ 371 Date: Dec. 1, 2010

(30) Foreign Application Priority Data
Feb. 25, 2008 (GB) 0803373.0

(51) Int. Cl. A61B 5/103; A61B 5/01

(52) U.S. Cl. 600/301; 600/306

(57) ABSTRACT

A spot or rash tester for meningitis (10) comprises a first transparent testing region (12) having a first profile and a second transparent testing region (14) having a second profile. The first profile is different to the second profile.

The present invention relates to a spot or rash tester.

Spots or rashes can be symptomatic of certain types of disease. It is a characteristic of some spots or rashes associated with particular illnesses, skin complaints or diseases that when pressure is applied to the spot or rash, its appearance changes. In this specification, the terms spot and rash can be used interchangeably. It is characteristic of rashes associated with other illnesses or diseases that when pressure is applied to the rash, it does not change in appearance. For example, it is known that for a meningococcal septicaemia or meningitis rash, when pressure is applied to the rash, it does not blanch. Such a rash is known as a non-blanching rash. The term “blanch” means to become white or pale or fade in this context—a rash that fades under pressure is said to blanch. Therefore it is known that a convenient test for indicating whether or not a rash is symptomatic of such a disease is to apply pressure to the rash and look for a change in appearance. In particular, adults or parents of children, possibly young children and infants, are advised to use a glass tumbler to apply pressure to a rash and to view the appearance of the rash through the glass tumbler in order to provide an indication as to whether a rash is symptomatic of, for example, meningococcal bacteria which causes meningitis and septicaemia, and immediate medical assessment is required

The present invention provides a spot or rash tester as claimed in claim 1.

Advantageously, a rash tester according to this invention comprises testing regions having different profiles. The profiles may be of a different size to each other. A smaller testing region may be more suitable for using upon young children and infants than a larger testing region. A smaller testing region may be more suitable for testing more enclosed areas, or cavity-like areas of a person's body (such as a neck or armpit). A larger testing region may be more suitable for older children or adults. Also, a larger testing area may be more suitable for testing other areas of the body, for example more open areas such as an arm or a leg.

The tester of this invention may include a body portion between the testing regions. When one of the testing regions is being used to test for a rash, the other testing region or the body portion or both the body portion and the other testing region may be used as a handle. This is advantageous over using a tumbler since a dedicated handle is provided and the tester is able to be more stably and consistently held against the skin. This may decrease the likelihood of breakage of the tester, since a curved glass tumbler can be difficult to handle, especially against small or enclosed body parts.

The tester of this invention comprises transparent testing regions. In this specification, a transparent testing region is one which is sufficiently transparent to enable a person using the tester to see the rash or spot underneath the testing region such that a determination can be made as to whether its appearance has changed or not to be indicative of a particular disease or other identifiable dermatological spot or rash. This may not require substantially complete transparency as in the case of clear glass, although in some embodiments such complete transparency may be present.

A tester according to this invention may be made from a glass material, or it may be made from a more suitable material, such as a plastics material. The tester of this invention may be made from a material which is less likely to shatter than glass when pressure is applied to it.

The testing regions may comprise curved or arcuate or smooth profiles.

Embodiments of the present invention will now be described by way of example only with reference to the accompanying drawings in which:

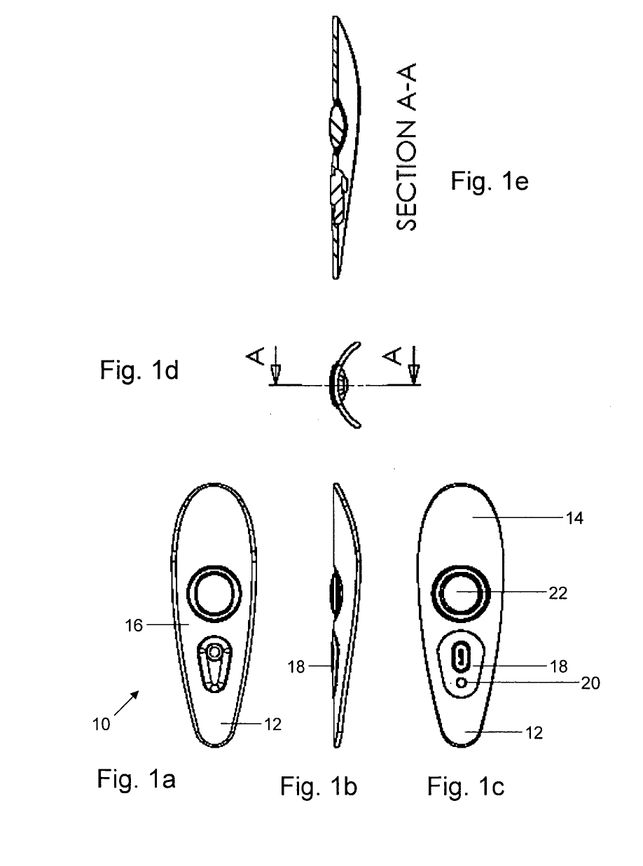

FIGS. 1a to 1e show a spot tester according to an embodiment of this invention—FIG. 1a is a view from underneath, FIG. 1b is a side view, FIG. 1c is a view from above, FIG. 1d is a view from an end, FIG. 1e is a sectional view from a side taken through the line A-A shown in FIG. 1d; and

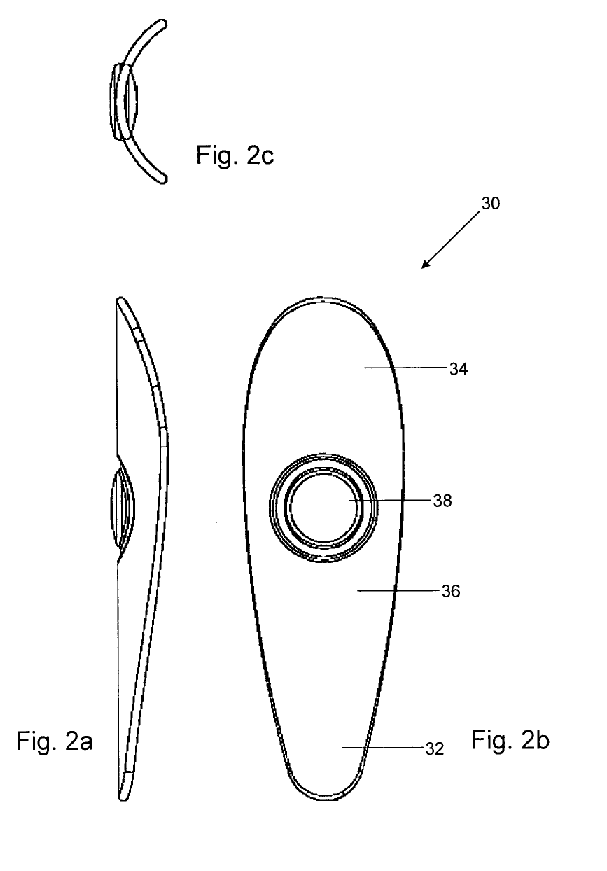

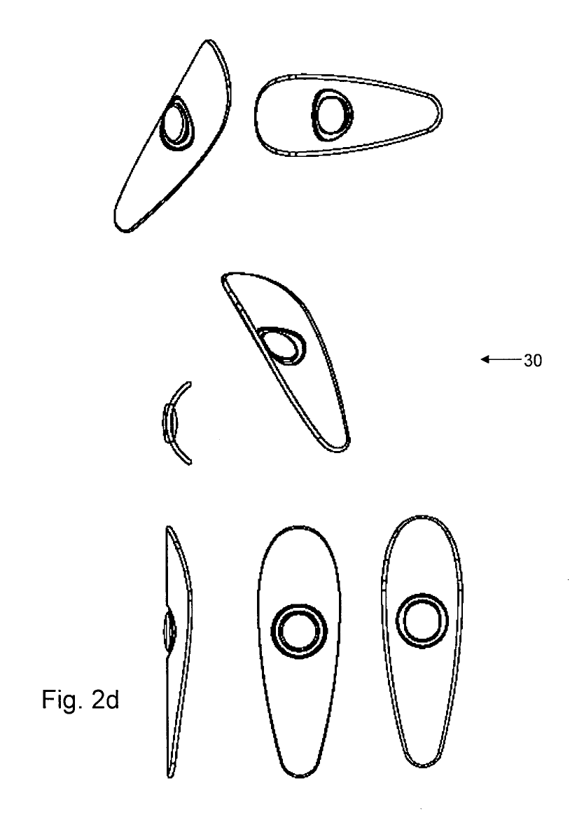

FIGS. 2a to 2d show a spot tester according to another embodiment—FIG. 2a is a side view, FIG. 2b is a view from above, FIG. 2c is an end view, FIG. 2d shows the views of FIGS. 2a to 2c and other perspective views.

Referring to FIGS. 1a to 1d, a spot tester 10 according to an embodiment of this invention comprises a first testing region 12 having a first profile and a second testing region 14 having a second profile. The first profile is different from the second profile.

In this embodiment the first profile is smaller than the second profile. In other embodiments the first and second profiles may be the same size. In this embodiment the first profile is of a different shape to the second profile. In other embodiments the first profile and the second profile may be of another shape, or they may be of the same shape or substantially the same shape as each other.

In this embodiment, the tester 10 also comprises a body portion 16 between the first testing region 12 and the second testing region 14. The body portion 16 is generally elongate and can be used to help securely grip and position the tester 10 when it is in use. The first testing region 12, second testing region 14 and body portion 16 are integrally formed from a transparent plastics material. The plastics material has anti-shatter properties to reduce the likelihood of the tester shattering in the case of excessive pressure being applied to it.

Both testing regions 12, 14 in this embodiment are substantially smooth, curved surfaces. In this embodiment edges of the testing regions are generally arcuate—the radius of curvature of the edge of the first testing region 12 is about 25 mm in this embodiment. The body portion 16 also has a curved profile. In this embodiment the curved testing regions 12, 14 are curved convexly away from the direction of the intended contact with skin of a person being tested. The tester 10 has a saddle-like shape. This shape does not have any protruding features, such as protruding edges or ridges which might stick into a person's body and cause discomfort while they are being tested. Therefore the testing regions 12, 14 are more easily used in tight spaces. In other embodiments, other shapes and sizes of testing regions can be used—such shapes and sizes will be apparent to the skilled person in view of the disclosure of this invention.

In use, the first testing region 12 is useful in testing spots in more difficult to access locations, such as in armpits or around the neck of an infant. The smaller, first testing region 12 is also more useful when testing the skin of infants or small children.

The larger second testing region 14 is useful in testing more open areas, such as an arm or a leg (of an infant or a larger child or adult). The second testing region 14 has a larger surface area, which may make the appearance of a spot or rash under pressure easier to see and so it can be preferable if the larger testing region 14 is used if possible. If it is not possible, then the first testing region 12 is a very useful alternative.

In other embodiments more than two testing regions may be provided—for example a tester having testing regions all of different shapes and sizes may be provided, or a tester having some testing regions of the same size, but different shapes (as appropriate to a particular body part) may be provided, or a tester having testing regions of the same shapes, but different sizes may be provided.

The tester may be between 6 cm and 20 cm long. The radius of curvature of the smaller testing region may be between 0.5 cm and 3 cm (for example 1 cm). The radius of curvature of the larger testing region may be between 1.5 cm and 10 cm (for example, 5 cm).

The tester 10 comprises a generally elongate tester so it is easily and comfortably held in the hand of a user. The length of the tester 10 is about 163 mm in this embodiment.

The tester 10 includes a thermometer 18. In this embodiment the thermometer is a contact thermometer arranged to measure the temperature of a person via contact with, for example, the forehead. The thermometer 18 is provided at the body portion 16 of the tester 10 so that it does not interfere with, or get damaged by, use of the testing portions 12, 14 when testing rashes.

In this embodiment the thermometer 18 is detachably attached to the tester 10. Attachment means 20 in the form of a snap-fit attachment is provided on the body portion 16 so that the thermometer 18 or a replacement thermometer 18 can be easily removed or added to the body portion 16, for example if it is required to be replaced following damage.

In other embodiments, the thermometer may be attached to different means, or at a different location on the tester 10. In yet further embodiments, the thermometer 18 may be integrally formed with the tester 10.

In some embodiments an infra-red thermometer is used. Infra-red thermometers provide quick body temperature readings. In other embodiments an electronic or digital thermometer may be used. The thermometer may be a tympanic thermometer or an axilla thermometer. In some embodiments the thermometer is integrally attached to the tester—in other embodiments the thermometer is detachable from the rest of the tester. In other embodiments the tester does not include a thermometer. The decision as to whether to use a digital or an infra-red thermometer may be based upon accuracy requirements.

The tester 10 also includes an integrally formed magnifier 22. The tester 10 and magnifier 22 are integrally formed from a transparent plastics material. The magnifier is used as a general purpose magnifier, e.g. to magnify moles or splinters or any other features of interest. In this way a very useful multi-purpose spot tester is provided.

In use, a person, for example a parent, selects an appropriate testing region 12 or 14 to test a spot on themselves, or on another person, such as a child. The testing region to be used is selected on the basis of the one that provides the most suitable shape or size or combination of shape and size or other profile characteristics for the particular body part of the particular person to be tested. Assuming the body part is a relatively open body part, such as a surface of the thigh, the individual or parent may select the larger testing region 14. As previously mentioned, this will have the extra advantage over the smaller testing region 12 of having a larger surface area over which to view the spot under pressure. However the smaller testing region can be used in this situation if favoured for some reason.

The individual or parent then presses the selected testing region 14 against the relevant spot or rash. If it is found that this testing region 14 is in fact too large, or of the wrong shape, then the other testing region 12 can be used. Similarly if the testing region 12 had been selected initially it is apparent that a larger or differently shaped testing region could be used, then the individual or parent may decide to switch from using the smaller testing region to using the larger testing region.

The appearance of the spot should be noted prior to pressing the testing region against it, and when the pressing action is performed, the spot should be monitored to check whether its appearance changes under pressure (e.g. whether it blanches, becomes white, pale or fades to usual skin colour) or whether its appearance does not significantly change to suggest non-blanching. From the result, a determination of whether or not a disease, such as meningococcal bacteria which causes meningitis and septicaemia, is suspected can be made. A combination of shape, size and material of the tester 10 makes it easier to handle than a glass tumbler, makes the pressing action more comfortable for the person pressing and the person being pressed and also provides a tester which is less likely to break or shatter due to excessive force being applied.

If the large testing region 14 is being used for testing, the small testing region 12, or the body portion 16, or a combination of both 12, 14 can be used as a handle due to their ergonomic profiles. As a result the tester 10 can be securely gripped without exerting undue excessive force.

Referring to FIGS. 2a to 2d, there is shown a spot tester 30 similar to the spot tester 10 in that it has first 32 and second 34 testing regions separated by a body portion 36. The tester 30 of this embodiment does not include a thermometer, but does include a magnifier 38.

In further embodiments the tester may include a thermometer but not a magnifier. In further embodiments the tester may include neither a thermometer nor a magnifier.

In one embodiment, the entire tester is formed of a plastic Fresnel lens material, which acts as a magnifier. The tester of this embodiment is flexible. Therefore all portions are provided with magnifying and viewing capability.

In some embodiments the tester comprises a camera arranged to capture an image at the or each or any one or more of the testing regions. There may be a camera designated to each testing region. In some embodiments there is a display provided on the tester. In some embodiments the camera readout is sent to a remote display—for example via a data transfer connection on the tester, such as a USB port provided on the tester. Other suitable forms of data transfer connection will be apparent to the skilled person. The data transfer may be wireless in some embodiments. In some embodiments the tester comprises a data capture button—this may be provided at or near or adjacent to one of the testing regions. In this way, the button should be conveniently located in an area which is held by a user when the tester is being used. More than one such button may be provided.

In some embodiments, a measurement mechanism is provided and arranged so that a user can objectively measure the size of a spot being tested. Usefully, the user can then check whether the spot is changing in size (growing or shrinking) over time. The measurement mechanism might be in the form of graduations provided on the tester at the, each, or any one or more of the testing regions. The graduations may be dots, notches or other such simple markings, or may be in the form of concentric circles, possibly centred at the centre of the relevant testing region.

In embodiments where there is both a camera and a measurement mechanism, it may be possible for the spot size to be determined automatically by a processor which is able to receive data associated with the camera readout.

Various modifications may be made to the present invention without departing from its scope. For example more than two testing regions may be provided in order to provide more combinations of shape, size or both. Different materials may be used to form the tester. The tester may not be integrally formed—different materials may be used to form different parts of the tester. A body portion may be differently shaped or sized. The body portion may not be present at all.

In some embodiments the tester comprises a magnifier arranged to magnify a region of interest, e.g. the or each testing region. In some embodiments the magnifier is provided integrally with the rest of the tester, for example by forming the region of interest from a different shape, dimensions or material such that the view of the region being tested is magnified and any spot or rash being tested can be more easily identified as being blanching or non-blanching. In another embodiment the magnifier comprises magnifying means which may be in the form of a magnifying window placed near or adjacent to the testing region to magnify the view of the testing region.

In other embodiments the tester comprises a magnifier arranged away from the region of interest. Such a magnifier may be used as a general purpose magnifier, e.g. to magnify moles or splinters or any other feature under inspection.

In some embodiments the tester comprises a light source arranged to illuminate the spot or rash of interest. The light source may comprise a battery operated light source. The light source may be operated completely or partially by solar power. The battery is preferably a small battery, e.g. a watch battery. The light source may be partially or completely operated by mains electricity power. The light source may be integrally attached to the tester, or in other embodiments may be removably attached to the tester. Illuminating the spot or rash may provide a better view of it such that a better judgement of whether it is blanching or non-blanching can be made.

1-24. (canceled)

25. A spot or rash tester comprising:
a transparent first testing region having a first profile; and
a transparent second testing region having a second profile different from the first profile and being connected to said first transparent testing region.

26. The tester of claim 25 wherein said first profile has a first size and said second profile has a relatively larger second size.

27. The tester of claim 25 said first profile has a first shape and said second profile has a second shape different from the first shape.

28. The tester of claim 25 wherein at least one of said first and second profiles is a smooth profile.

29. The tester of claim 25 said first profile has at least one of a width between 0.5 cm and 3 cm and a length between 0.5 cm and 5 cm.

30. The tester of claim 25 wherein said second profile has at least one of a width between 1.5 cm and 10 cm and a length between 1.5 cm and 20 cm.

31. The tester of claim 25 including a body portion connecting said first testing region with said second testing region.

32. The tester of claim 31 wherein said body portion has an elongate shape.

33. The tester of claim 31 wherein at least one of said second testing region and said body portion is formed as a handle for grasping by a person when said first testing region is used for testing and wherein at least one of said first testing region and said body portion is formed as a handle for grasping by a person when said second testing region is used for testing.

34. The tester of claim 25 having an elongate shape extending between said first and second testing regions.

35. The tester of claim 25 wherein at least a portion of the tester is formed from an anti-shatter material.

36. The tester of claim 25 wherein at least a portion of the tester is formed from at least one of an acetal plastic material and a polycarbonate material.

37. The tester of claim 25 including a thermometer mounted adjacent said first and second testing regions.

38. The tester of claim 37 including a body portion connecting said first testing region with said second testing region and wherein said thermometer is mounted on said body portion.

39. The tester of claim 37 wherein said thermometer is selectively removable from the tester.

40. The tester of claim 37 including a body portion connecting said first testing region with said second testing region and including attachment means on said body portion for removably attaching said thermometer.

41. The tester of claim 25 including magnifying means arranged to magnify a view through at least one of said first testing region and said second testing region.

42. The tester of claim 25 including at least one of a light source arranged to illuminate a spot or a rash being tested and a camera arranged to capture an image of the spot or the rash being tested.

43. The tester of claim 42 including a camera arranged to capture an image of a spot or a rash being tested and a data capture button for actuation to transfer the image from said camera.

44. The tester of claim 25 including a measurement mechanism arranged to facilitate measurement of a size of a spot or a rash being tested.
